(19) United States
(12) Patent Application Publication
(10) Pub. No.: US 2016/0124363 A1
(43) Pub. Date: May 5, 2016

(54) SHEET LENGTH MEASURING APPARATUS AND IMAGE FORMING APPARATUS

(71) Applicant: Nozomu TAKAHASHI, Ibaraki (JP)

(72) Inventor: Nozomu TAKAHASHI, Ibaraki (JP)

(21) Appl. No.: 14/887,937

(22) Filed: Oct. 20, 2015

(30) Foreign Application Priority Data
Nov. 4, 2014 (JP) 2014-224724

(51) Int. Cl. G03G 15/00; G01N 33/34; G01B 11/02

(52) U.S. Cl. CPC G03G 15/5029; G01B 11/02; G01N 33/346

(57) ABSTRACT

A sheet length measuring apparatus includes first and second detection units respectively provided downstream and upstream of a conveyance unit, each detection unit detecting passage of an edge of a sheet medium; a first control unit to, until a predetermined set time passes since the detection of the passage of a leading edge of the sheet medium, regard detection of the passage of an edge of the sheet medium by each of the first and second detection units as not detected; and a second control unit to, when passage of an edge of the sheet medium is not detected again before a lapse of a predetermined monitoring time since the second detection unit detects passage of an edge of the sheet medium being passing a detection position of the second detection unit, judge that the edge detected by the second detection unit is a trailing edge of the sheet medium.

### CROSS-REFERENCE TO RELATED APPLICATIONS

The present application claims priority to and incorporates by reference the entire contents of Japanese Patent Application No. 2014-224724 filed in Japan on Nov. 4, 2014.

### BACKGROUND OF THE INVENTION

1. Field of the Invention

The present invention relates to a sheet length measuring apparatus and an image forming apparatus.

2. Description of the Related Art

In the commercial printing industry, a transition from known offset printing to POD (Print on Demand) with an image forming apparatus using the electrophotographic system is in progress for small-lot, various types of, and variable-data printing. In the image forming apparatus using the electrophotographic system, the accuracy of registration between the front side and back side of a sheet (a recordable sheet medium) comparable to an offset printer is coming to be required to deal with such needs.

Factors of misregistration between the front side and back side of a sheet can be roughly divided into a registration error in the vertical direction/horizontal direction, and a skew error of a sheet and an image. However, in an image forming apparatus with a heat fixing device, an image scaling error due to the expansion/contraction of a sheet is added. Hence, a technology is expected in which a front and a back image scaling factor are determined based on a precalculated amount of expansion/contraction of a sheet to correct image sizes, and accordingly the image scaling error on both sides of the sheet is reduced.

To automatically correct the image scaling error (image scaling correction) on both sides of the sheet, a technology for measuring a sheet size is required. Hence, disclosed are a technology for detecting a leading end and a trailing end of a sheet to be conveyed with sensors, and measuring the length of the sheet from the number of pulses counted during a sheet passage time, and a technology for measuring the length of a sheet from the number of pulses of a rotary encoder on a sheet conveyance roller shaft (see, for example, Japanese Patent Application Laid-open No. 2013-053004).

The position of a hole in special paper having a punched hole or the like, for example, a punched sheet and a sheet with a locally bored hole, is arbitrary. Specifying the position of the hole narrows the degree of freedom in a user's selection of a sheet, and is difficult.

Therefore, in the sheet length measuring technology of the known image forming apparatus, if a portion having a hole in a sheet such as a punched hole passes a sensing position of the sensor that detects a leading or trailing end of a sheet, an edge of the portion having the hole may be wrongly detected as an edge (edge) of the sheet. If the edge of the sheet is wrongly detected in this manner, the length of the sheet is wrongly measured. Accordingly, there is a problem in the above-mentioned special paper having a hole that the image scaling correction cannot be made.

Therefore, there is a need for a sheet length measuring apparatus and image forming apparatus that enables even the measurement of a sheet length of a special sheet medium having a hole such as a punched hole, and can make the image scaling correction.

### SUMMARY OF THE INVENTION

It is an object of the present invention to at least partially solve the problems in the conventional technology.

According to an embodiment, a sheet length measuring apparatus includes a conveyance unit, a first detection unit, a second detection unit, a conveyance amount measuring unit, a calculation unit, a first control unit, and a second control unit. The conveyance unit conveys a sheet medium. The first detection unit is provided downstream of the conveyance unit in a conveyance direction of the sheet medium to detect passage of an edge of the sheet medium. The second detection unit is provided upstream of the conveyance unit in the conveyance direction to detect passage of an edge of the sheet medium. The conveyance amount measuring unit measures a conveyance amount of the sheet medium conveyed by the conveyance unit. The calculation unit calculates a length of the sheet medium in the conveyance direction from detection results of the first and second detection units and the conveyance amount of the sheet medium. Until a predetermined set time passes since the detection of the passage of a leading edge in the conveyance direction among edges of the sheet medium, the first control unit regards detection of the passage of an edge of the sheet medium by each of the first and second detection units as not detected. When passage of an edge of the sheet medium is not detected again before a lapse of a predetermined monitoring time since the second detection unit detects passage of an edge of the sheet medium being passing a detection position of the second detection unit, the second control unit judge that the edge detected by the second detection unit is a trailing edge in the conveyance direction among the edges of the sheet medium.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an example of the configuration of an image forming apparatus according to an embodiment;

FIG. 2 is a schematic diagram (plan view) illustrating a mechanical configuration example of a sheet length measuring apparatus according to an embodiment;

FIG. 3 is a schematic diagram (side view) illustrating the mechanical configuration example of the sheet length measuring apparatus according to an embodiment;

FIG. 4 is a block diagram illustrating a functional configuration example of the sheet length measuring apparatus according to an embodiment;

FIG. 5 is an explanatory diagram of a start timing and an end timing of a pulse signal count according to an embodiment; and

FIG. 6 is a timing chart related to a pulse count at the time when an image is printed on a sheet in the image forming apparatus according to an embodiment.

### DETAILED DESCRIPTION OF THE PREFERRED EMBODIMENTS

An embodiment of a sheet length measuring apparatus and an image forming apparatus is described hereinafter in detail with reference to the accompanying drawings. Firstly, a description is given below of the configuration of an image forming apparatus with a sheet length measuring apparatus according to the embodiment. The embodiment is described taking a sheet of paper (hereinafter, simply referred to as a sheet) as an example of a sheet medium that is recordable by the image forming apparatus. However, as long as it is a recordable medium, another medium can be applied.

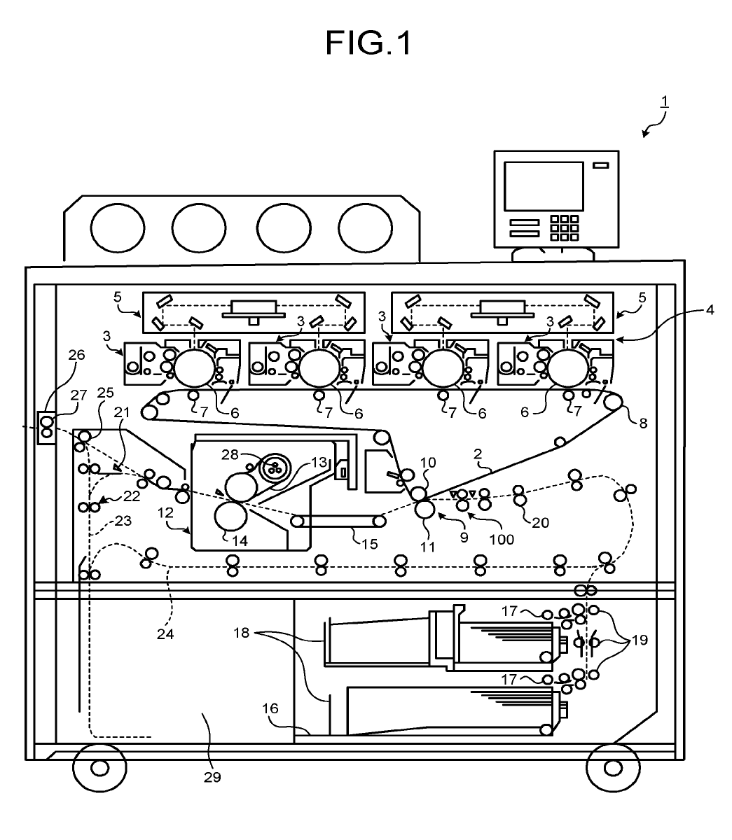

FIG. 1 is a schematic diagram illustrating an example of the configuration of an image forming apparatus according to an embodiment. As illustrated in FIG. 1, an image forming apparatus 1 is provided with an endless belt type intermediate transfer belt 2 near its center. The intermediate transfer belt 2 is looped over a plurality of support rollers, and is configured to be capable of rotation and conveyance in a clockwise direction in the figure.

A plurality of image forming units 3 is arranged side by side above the intermediate transfer belt 2 along a conveyance direction of the intermediate transfer belt 2 to form a tandem image forming unit 4. Furthermore, an exposure device 5 is provided above the tandem image forming unit 4.

A photoconductor drum 6 as an image bearer that bears each color toner image is provided to each image forming unit 3 of the tandem image forming unit 4. Moreover, a primary transfer roller 7 is provided, opposed to each photoconductor drum 6 across the intermediate transfer belt 2, at a primary transfer position where a toner image is transferred from the photoconductor drum 6 onto the intermediate transfer belt 2. Moreover, a support roller 8 is a drive roller that drives and rotates the intermediate transfer belt 2.

A secondary transfer unit 9 is provided opposite to the tandem image forming unit 4 across the intermediate transfer belt 2. The secondary transfer unit 9 presses a secondary transfer roller 11 against a secondary transfer counter roller 10 to apply a transfer electric field. Accordingly, an image on the intermediate transfer belt 2 is transferred onto a sheet P (a sheet medium) (not illustrated). In the secondary transfer unit 9, a transfer current of the secondary transfer roller 11 being a parameter of a transfer condition is changed depending on the sheet P.

A sheet length measuring apparatus 100 is provided upstream of the secondary transfer unit 9. Moreover, a fixing unit 12 that thermally fuses and fixes the transferred image (toner image) on the sheet P is provided downstream of the secondary transfer unit 9. The sheet length measuring apparatus 100 measures a sheet length L (the length of the sheet P) before and after passing the fixing unit 12 upon duplex printing, and corrects an image scaling error (the image scaling correction) on a back side of the sheet based on the sheet expansion/contraction rate.

The fixing unit 12 includes a halogen lamp 28 as a heat source, and is configured in such a manner as that a pressure roller 14 is pressed against a fixing belt 13 being an endless belt. The fixing unit 12 changes the temperatures of the fixing belt 13 and the pressure roller 14, a nip width between the fixing belt 13 and the pressure roller 14, and the speed of the pressure roller 14, which are parameters of fixing conditions, depending on the sheet P. The sheet P after image transfer is then conveyed to the fixing unit 12 by a conveyance belt 15.

Here, a description is given of the flow until the time when an image is printed by the image forming apparatus 1 on the sheet P. In the image forming apparatus 1, when image data is received first to accept a signal to start image formation, the support roller 8 is driven and rotated by a drive motor (not illustrated) to drive and rotate a plurality of other support rollers. Accordingly, the intermediate transfer belt 2 is rotated and conveyed. At the same time, a single-colored image is formed on each photoconductor drum 6 in each image forming unit 3. The formed single-colored images are sequentially transferred by the primary transfer rollers 7 with the conveyance of the intermediate transfer belt 2 to form a composite color image on the intermediate transfer belt 2.

Moreover, one of paper feeding rollers 17 of a paper feeding table 16 is selected and rotated. The sheet P is sent out of one of paper feeding cassettes 18, conveyed by a conveyance roller 19, and caused to abut a registration roller 20 to stop. The registration roller 20 is rotated timed to the composite color image on the intermediate transfer belt 2 to transfer the image at the secondary transfer unit 9. Accordingly, the color image is recorded on the sheet P.

The sheet P after image transfer is conveyed by the secondary transfer unit 9 to be sent to the fixing unit 12. In the fixing unit 12, heat and pressure are applied to fuse and fix the transferred image. In a case of duplex printing, a bifurcating claw 21 and a flip roller 22 convey the sheet P to a sheet reversal path 23 and a duplex conveyance path 24 to record a composite color image also on a back side of the sheet P as in the above-mentioned recording on the front side of the sheet P.

Moreover, if the sheet P is reversed, the sheet P is conveyed by the bifurcating claw 21 to the sheet reversal path 23 and conveyed by the flip roller 22 to a paper ejection roller 25 side. Accordingly, the sheet P is turned upside down. In cases of simplex printing and no paper reversal, the sheet P is conveyed by the bifurcating claw 21 to the paper ejection roller 25. The sheet P is then conveyed by the paper ejection roller 25 to a decurler unit 26. A decurling amount is changed depending on the sheet P in the decurler unit 26. The pressure of a decurling roller 27 is changed to adjust the decurling amount. The sheet P is ejected by the decurling roller 27.

A purge tray 29 is placed below a paper reversal/ejection unit. In the image forming apparatus 1, eight sheets are conveyed, including the reversal path, during continuous duplex printing. A new sheet P is fed from the paper feeding cassette 18 to between the fourth sheet with a printed front side and the fifth sheet with a printed front side.

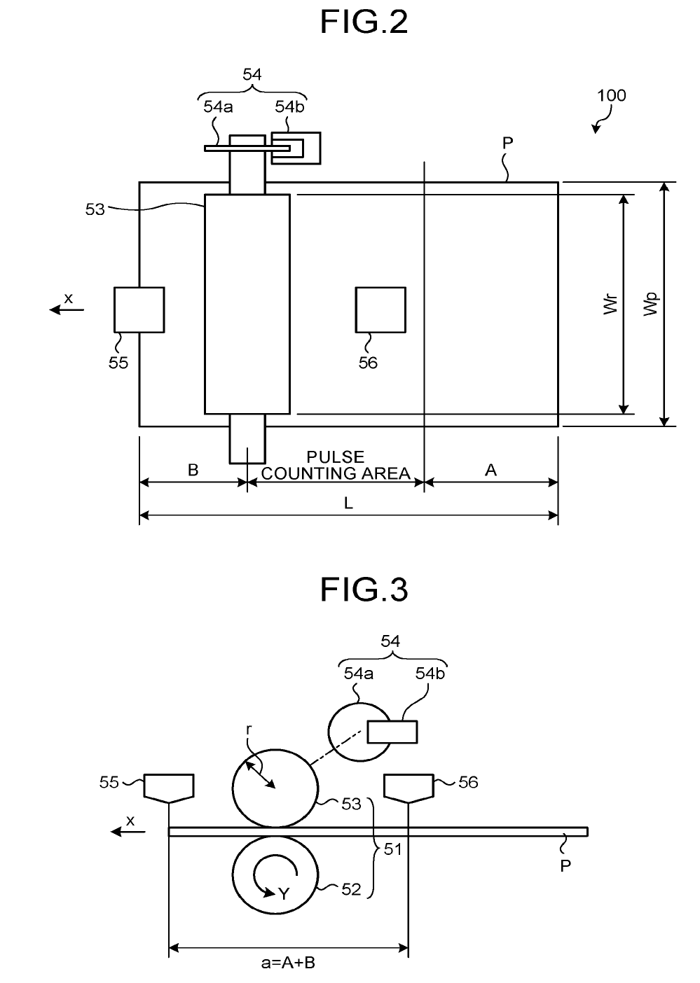

Next, the configuration of the sheet length measuring apparatus 100 according to the embodiment is described. FIGS. 2 and 3 are schematic diagrams illustrating a mechanical configuration example of the sheet length measuring apparatus according to an embodiment. FIG. 2 is a plan view of the sheet length measuring apparatus when viewed from an upper side of the sheet P. FIG. 3 is a side view of the sheet length measuring apparatus.

The sheet length measuring apparatus 100 according to the embodiment measures the sheet length L being a length in the conveyance direction of the sheet P to be conveyed along a predetermined conveyance path. A conveyance roller (a conveyance unit) as a rotary body that rotates with the conveyance of the sheet P is provided in the conveyance path of the sheet P. The conveyance roller 51 of the embodiment is configured as a pair of rollers that is a combination of a drive roller 52 and a driven roller 53. The drive roller 52 and the driven roller 53, which constitute the conveyance roller 51, rotate while sandwiching the sheet P to convey the sheet P in an X direction in FIGS. 2 and 3.

The sheet P is conveyed by another conveyance unit (not illustrated) in the X direction in FIGS. 2 and 3 before its leading edge reaches the position of the conveyance roller 51 and after its trailing edge passes the position of the conveyance roller 51. The leading edge of the sheet P is an edge of a leading end in the conveyance direction among edges of the sheet. Moreover, the trailing edge of the sheet P is an edge of a trailing end in the conveyance direction among the edges of the sheet.

The drive roller 52 is configured to be driven and rotated in a Y direction in FIG. 3 by a motor (not illustrated) and power transmission units (for example, a gear and belt). The drive roller 52 includes a rubber layer on the surface to generate a sufficient friction force, for example, in between with the sheet P.

The driven roller 53 is disposed in such a manner as to apply pressure to and come into contact with the drive roller 52 side by a biasing unit (for example, a spring) (not illustrated). The driven roller 53 is rotated in contact with the drive roller 52 when not conveying the sheet P (during idling), and is rotated by the friction force generated in between with the sheet P when conveying the sheet P. The driven roller 53 is configured as, for example, a metal roller to ensure the accuracy of axial runout.

As illustrated in FIG. 2, a length Wr of the driven roller 53 in a width direction orthogonal to the conveyance direction of the sheet P is smaller than a minimum value of a width Wp of the sheet P to be handled by the sheet length measuring apparatus 100. Therefore, the driven roller 53 is not in contact with the drive roller 52 and is rotated only by the friction force generated in between with the sheet P, when conveying the sheet P.

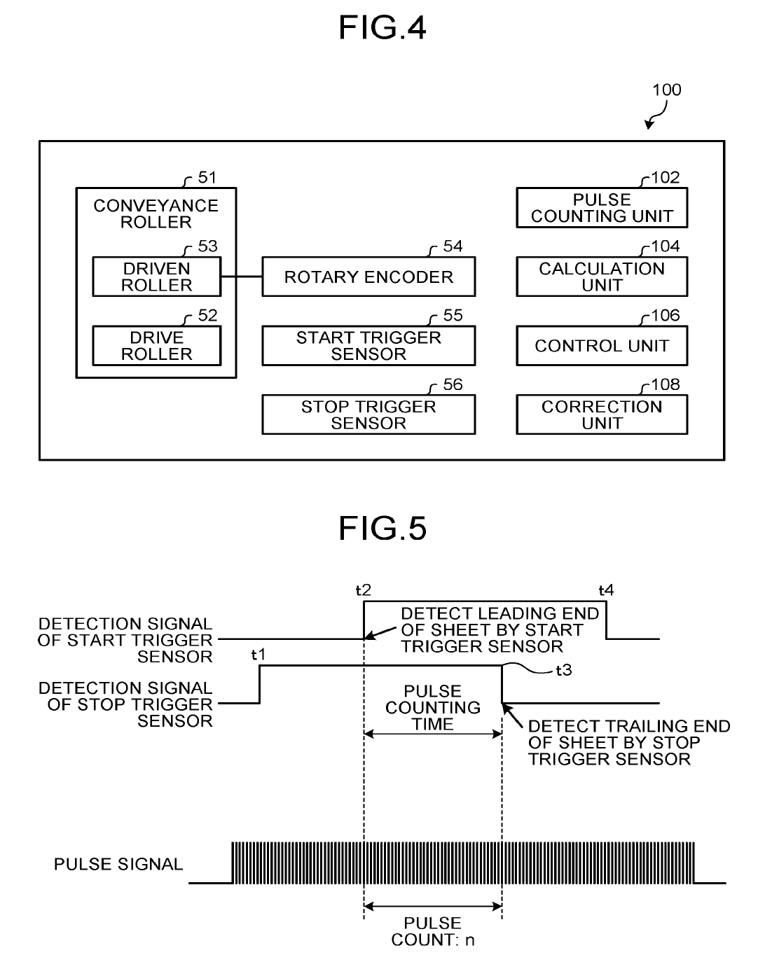

A rotary encoder 54 is provided on a rotation shaft of the driven roller 53. The rotary encoder 54 includes an encoder disc 54a that rotates integrally with the driven roller 53, and an encoder sensor 54b that generates a pulse in accordance with the rotation of the encoder disc 54a. The rotary encoder 54 outputs a pulse signal when the driven roller 53 is rotated. The pulse signal output by the rotary encoder 54 is input to a pulse counting unit 102 (see FIG. 4) described below. It may be a configuration where some device that outputs a signal in accordance with the rotation amount of the driven roller 53 is provided instead of the rotary encoder 54.

In the examples illustrated in FIGS. 2 and 3, the driven roller 53 is placed on the upper side of the sheet P. The drive roller 52 is placed on the back side of the sheet P. The positions of the drive roller 52 and the driven roller 53 may be the other way around. Moreover, the conveyance roller 51 included in the sheet length measuring apparatus 100 may be configured to rotate with the conveyance of the sheet P. In other words, the conveyance roller 51 is not necessarily configured as the pair of rollers being a combination of the drive roller 52 and the driven roller 53. The conveyance roller 51 may be configured of a single roller. Furthermore, the conveyance roller 51 may have a configuration where, for example, a spherical rotary body is provided instead of the roller-shaped conveyance roller 51.

A start trigger sensor 55 (a first detection unit) that detects the passage of an edge of the sheet P to be conveyed is provided downstream of the conveyance roller 51 (the drive roller 52 and the driven roller 53) in the conveyance direction of the sheet P. Moreover, a stop trigger sensor 56 (a second detection unit) that detects the passage of an edge of the sheet P to be conveyed is provided upstream of the conveyance roller 51 (the drive roller 52 and the driven roller 53) in the conveyance direction of the sheet P.

The sheet length measuring apparatus 100 of the embodiment starts counting pulses when the leading edge of the conveyed sheet P passes the start trigger sensor 55 provided downstream of the conveyance roller 51. Next, the pulse count ends when the trailing edge of the conveyed sheet P passes the stop trigger sensor 56 provided upstream of the conveyance roller 51. Subsequent sheets P are also similarly conveyed to count pulses.

For example, transmissive or reflective optical sensors having high accuracy in detecting an edge of the sheet P can be used for the start trigger sensor 55 and the stop trigger sensor 56, which are provided in the conveyance path of the sheet P. In the embodiment, reflective optical sensors are used for the start trigger sensor 55 and the stop trigger sensor 56.

In the example illustrated in FIGS. 2 and 3, the start trigger sensor 55 and the stop trigger sensor 56 are placed on the upper side of the sheet P. However, both or either of the start trigger sensor 55 and the stop trigger sensor 56 may be placed on the back side of the sheet P.

A distance A illustrated in FIG. 2 is a distance between the stop trigger sensor 56 and the conveyance roller 51 (the drive roller 52 and the driven roller 53). A distance B illustrated in FIG. 2 is a distance between the start trigger sensor 55 and the conveyance roller 51 (the drive roller 52 and the driven roller 53). A distance a (see FIG. 3) that is a value obtained by adding the distances A and B is a distance between the start trigger sensor 55 and the stop trigger sensor 56. The distance a is shorter than a minimum value of the length of a sheet that can be measured by the sheet length measuring apparatus 100.

FIG. 4 is a block diagram illustrating a functional configuration example of the sheet length measuring apparatus according to an embodiment. As illustrated in FIG. 4, the sheet length measuring apparatus 100 mainly includes the conveyance roller 51 with the drive roller 52 and the driven roller 53, the rotary encoder 54, the start trigger sensor 55, the stop trigger sensor 56, the pulse counting unit 102, a calculation unit 104, a control unit 106, and a correction unit 108. The descriptions of the conveyance roller 51, the rotary encoder 54, the start trigger sensor 55, and the stop trigger sensor 56 are omitted since they were described in FIGS. 2 and 3.

The pulse counting unit 102 is for measuring the conveyance amount of the sheet P to be conveyed by the conveyance roller 51 being the conveyance unit, and corresponds to a conveyance amount measuring unit. Specifically, the pulse counting unit 102 counts pulse signals generated by the encoder sensor 54b by the rotation of the encoder disc 54a of the rotary encoder 54 provided to the driven roller 53. Accordingly, the pulse counting unit 102 measures the conveyance amount of the sheet P.

Here, a description is given of a start timing and an end timing of the pulse signal count by the pulse counting unit 102. FIG. 5 is an explanatory diagram of a start timing and an end timing of a pulse signal count according to an embodiment.

As described above, when the driven roller 53 rotates, a pulse signal is generated from the rotary encoder 54 provided on the rotation shaft of the driven roller 53. The sheet P is conveyed by the conveyance roller 51. After the stop trigger sensor 56 detects the passage of the leading edge of the sheet P at time t1 in FIG. 5, the start trigger sensor 55 detects the passage of the leading edge of the sheet P at time t2 in FIG. 5.

Next, after the stop trigger sensor 56 detects the passage of the trailing edge of the sheet P at time t3 in FIG. 5, the start trigger sensor 55 detects the passage of the trailing edge of the sheet P at time t4 in FIG. 5.

At the timing when the start trigger sensor 55 detects the passage of the leading edge of the sheet P at time t2 in FIG. 5, the pulse counting unit 102 starts counting pulse signals (the pulse count) generated from the rotary encoder 54. At the timing when the start trigger sensor 55 detects the passage of the trailing edge of the sheet P at time t3, the pulse counting unit 102 ends the count of pulses of the rotary encoder 54. In other words, from time t2 when the start trigger sensor 55 detects the passage of the leading edge of the sheet P to time t3 when the stop trigger sensor 56 detects the passage of the trailing edge of the sheet P is the time to count pulse signals (the pulse counting time).

The calculation unit 104 calculates the length L of the sheet P in the conveyance direction (the sheet length L) from the detection results of the sheet P by the start trigger sensor 55 and the stop trigger sensor 56, and the conveyance amount of the sheet P.

A method for calculating the sheet length L (the length of the sheet P) is described below. The radius of the driven roller 53 provided with the rotary encoder 54 is denoted by r (See FIG. 3), the number of encoder pulses per rotation of the driven roller 53 by N, the number of pulses counted during the pulse counting time by n, and the distance between the start trigger sensor 55 and the stop trigger sensor 56, which determine the start and the end of the pulse count, by a. The sheet length L is obtained by the following equation (1).

L=(n/N)×2πr+a (1)

n: the obtained pulse count
N: the number of encoder pulses per rotation of the driven roller 53 [/r]
r: the radius of the driven roller 53 [mm]
a: the distance between the start trigger sensor 55 and the stop trigger sensor 56 [mm]

Here, the relationship between the conveyance speed of the sheet P (the sheet conveyance speed) and the sheet length L is described. Generally, the sheet conveyance speed changes depending on the outer dimension accuracy of the conveyance roller 51 (especially the drive roller 52), the machine precision such as the accuracy of axial runout, the rotation accuracy of a motor and the like, and the power transmission accuracy of a gear, belt, and the like. Moreover, the sheet conveyance speed also changes due to a slip between the drive roller 52 and the sheet P, a slack caused by a difference between upstream and downstream sheet conveyance forces or sheet conveyance speeds in the conveyance roller 51.

Hence, the pulse cycle and pulse width of the rotary encoder 54 change, but the number of pulses does not change. Therefore, the calculation unit 104 of the sheet length measuring apparatus 100 can calculate the sheet length L by the equation (1) with high accuracy without depending on the sheet conveyance speed.

The calculation unit 104 can also obtain a relative ratio such as the ratio between pages of the sheets P and the ratio of the front side to the back side. For example, the calculation unit 104 can obtain an expansion/contraction rate R by the following equation (2) from the relative ratio of the sheet length L before and after the heat fixing in the electrophotographic system.

R=[(n2/N)×2πr+a]/[(n1/N)×2πr+a]≈n2/n1 (2)

n1: the number of pulses counted when the sheet P passes before heat fixing
n2: the number of pulses counted when the sheet P passes after heat fixing

If the distance a to determine the start and the end of the pulse count (the distance between the start trigger sensor 55 and the stop trigger sensor 56) is sufficiently smaller than the sheet length L, the relative ratio of the sheet length L approaches the rate of the counted number of pulses. Moreover, the distance a is determined based on a sheet size supported by the image forming apparatus 1 and based on the sheet length measurement accuracy required by the image forming apparatus 1. However, in the embodiment, the distance a is determined to be equal to or less than one-tenth of the maximum sheet length.

Until a predetermined set time passes since the detection of the passage of the leading edge of the sheet P, the control unit 106 enters a masked state in which the control unit 106 regards the detection of the passage of an edge of the sheet P by each of the start trigger sensor 55 and the stop trigger sensor 56 as not detected. In such a masked state, even if the start trigger sensor 55 and the stop trigger sensor 56 actually detect an edge of the sheet P, the control unit 106 performs a process similar to a process in a state where there is no detection.

The control unit 106 can set the above-mentioned predetermined set time to an arbitrary time. Hence, the sheet length measuring apparatus 100 of the embodiment can appropriately set a mask time for the start trigger sensor 55 and the stop trigger sensor 56 in accordance with the conveyance speed of the sheet P and the sheet length L. In the embodiment, the control unit 106 can set the predetermined set time to a time from after the detection of the passage of the leading edge of the sheet P to before the passage of the trailing edge of the sheet P. Consequently, a hole portion such as a punched hole in the sheet P cannot be wrongly detected as the trailing edge of the sheet P within the predetermined set time.

The control unit 106 judges whether or not the passage of an edge of the sheet P was detected again before a lapse of a predetermined monitoring time since the stop trigger sensor 56 detected the passage of an edge of the sheet P being passing the detection position (sensing position) of the stop trigger sensor 56. If the passage of an edge of the sheet P was not detected again before a lapse of the predetermined monitoring time, the control unit 106 judges that the edge detected by the stop trigger sensor 56 is the trailing edge of the sheet P. If the passage of an edge of the sheet P was detected at least one time or more before a lapse of the predetermined monitoring time, the control unit 106 judges that the edge lastly detected by the stop trigger sensor 56 is the trailing edge of the sheet P.

In this case, the control unit 106 can set the above-mentioned monitoring time to an arbitrary time. Hence, the sheet length measuring apparatus 100 of the embodiment can appropriately set the monitoring time for the start trigger sensor 55 and the stop trigger sensor 56 in accordance with the conveyance speed of the sheet P and the sheet length L. In the embodiment, the control unit 106 sets the monitoring time to a time from after the detection of the passage of an edge of the sheet P being passing the detection position of the stop trigger sensor 56 to the passage of the diameter of the hole portion in the sheet P. Consequently, it is possible to judge whether the detected edge of the sheet P is the trailing edge or a rear portion. The control unit 106 corresponds to a first control unit and a second control unit.

The correction unit 108 corrects the image scaling error (the image scaling correction) of a toner image to be recorded on the sheet P, based on the measured front to back ratio of the sheet length L. A publicly known method can be used for the image scaling correction. As a result, the sizes of images recorded on the front and back sides of the sheet P agree with each other to improve the accuracy of registration between the front side and the back side.

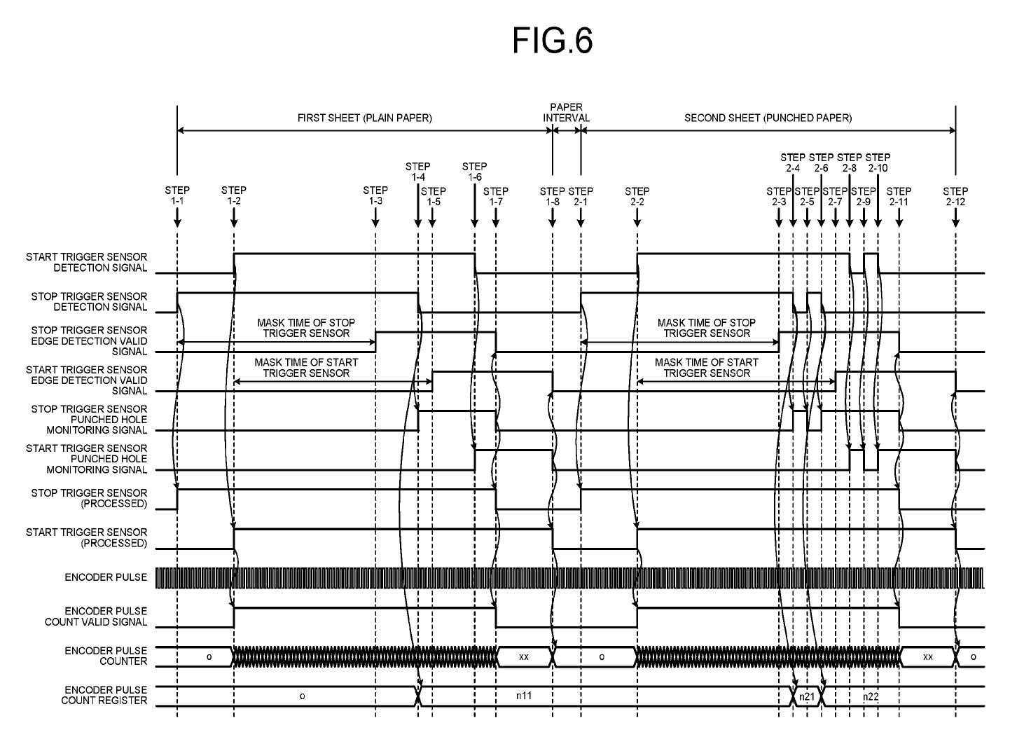

FIG. 6 is a timing chart related to a pulse count at the time when an image is printed on a sheet in the image forming apparatus according to an embodiment. FIG. 6 illustrates a case where an image is printed on a first sheet of plain paper, and a case where an image is printed on a second sheet of punched paper being a type of special paper.

In terms of the second sheet of punched paper of FIG. 6, the punched hole passes the sensing positions (detection positions) of the start trigger sensor 55 and the stop trigger sensor 56. Moreover, in FIG. 6, a start trigger sensor detection signal is a signal to be output by the start trigger sensor 55, and a stop trigger sensor detection signal is a signal to be output by the stop trigger sensor 56. Moreover, a stop trigger sensor edge detection valid signal, a start trigger sensor edge detection valid signal, a stop trigger sensor punched hole monitoring signal, a start trigger sensor punched hole monitoring signal, a signal of the stop trigger sensor (processed), and a signal of the start trigger sensor (processed) are processing signals to be internally used by the control unit 106, and are not output to the outside.

Firstly, the case where the first sheet of plain paper is conveyed is described with reference to FIG. 6. When the stop trigger sensor detection signal is asserted (put into a signal valid state), the control unit 106 latches the signal of the stop trigger sensor (processed) to mask detection by the stop trigger sensor 56 until the predetermined set time set depending on the print speed and the sheet length L. In the embodiment, the mask time of detection by the stop trigger sensor 56 is set in such a manner as to unmask at a point of 10 mm in the sheet length L (STEP 1-1). The signal of the stop trigger sensor (processed) indicates a signal from which a level changed portion of a sensor signal (sensor signals output by the stop trigger sensor 56 and the start trigger sensor 55) caused by the punched hole has been eliminated.

Next, when the start trigger sensor detection signal is asserted, the control unit 106 latches the signal of the start trigger sensor (processed) to mask detection by the start trigger sensor 55 until the predetermined set time set depending on the print speed and the sheet length L. When the signal of the start trigger sensor (processed) is asserted and latched, the control unit 106 operates an encoder pulse counter, and the pulse counting unit 102 starts counting pulse signals. In the embodiment, the mask time of detection by the start trigger sensor 55 is set in such a manner as to unmask at the point of 10 mm in the sheet length L (STEP 1-2). The encoder pulse counter is a counter that counts pulse signals output by the encoder disc 54a and the encoder sensor 54b.

Next, after a lapse of the set mask time (the time from masking to unmasking) of the stop trigger sensor 56, the control unit 106 asserts the stop trigger sensor edge detection valid signal, and enters a negate detection valid state in which the stop trigger sensor 56 can detect the passage of an edge of the sheet P (STEP 1-3).

When a negate (a signal invalid state) at an edge of the sheet P is detected by the stop trigger sensor detection signal, the control unit 106 stores a count value of the encoder pulse counter in an encoder pulse count register (the count value at this point in time is n11). In order to judge whether the negate is the trailing edge of the sheet P or an edge of the punched hole, the control unit 106 sets, as the monitoring time, the time required by the diameter of the punched hole to pass the stop trigger sensor 56, and asserts and monitors the stop trigger sensor punched hole monitoring signal (STEP 1-4). The encoder pulse count register is a register (not illustrated) that stores the pulse counting result.

Next, after a lapse of the set mask time of the start trigger sensor 55, the control unit 106 asserts the start trigger sensor edge detection valid signal, and enters the negate detection valid state in which the start trigger sensor 55 can detect the passage of an edge of the sheet P (STEP 1-5).

When the negate at an edge of the sheet P is detected by the start trigger sensor detection signal, in order to judge whether the negate is the trailing edge of the sheet P or an edge of the punched hole, the control unit 106 sets, as the monitoring time, the time required by the diameter of the punched hole to pass the start trigger sensor 55, and asserts and monitors the start trigger sensor punched hole monitoring signal (STEP 1-6).

If an edge of the sheet P is not detected within the set monitoring time, the control unit 106 negates the stop trigger sensor punched hole monitoring signal, and judges that the edge of the sheet P detected in STEP 1-4 is the trailing edge of the sheet P. The control unit 106 then negates the signal of the stop trigger sensor (processed), and negates the stop trigger sensor edge detection valid signal. When the signal of the stop trigger sensor (processed) is negated, the encoder pulse count valid signal is negated to stop the encoder pulse counting (STEP 1-7). The encoder pulse count valid signal is a signal that allows the counting of pulse signals, and counts pulse signals only while the signal is being enabled.

After a lapse of the set monitoring time, the control unit 106 negates the start trigger sensor punched hole monitoring signal, negates the signal of the start trigger sensor (processed), and negates the start trigger sensor edge detection valid signal. When the signal of the start trigger sensor (processed) is negated, the encoder pulse counter is cleared to an initial state (the initial value of the embodiment is zero) (STEP 1-8).

Next, the case where the second sheet of punched paper is conveyed is described. When the stop trigger sensor detection signal is asserted, the control unit 106 latches the signal of the stop trigger sensor (processed) to mask detection by the stop trigger sensor 56 until the predetermined set time set depending on the print speed and the sheet length L. In the embodiment, the mask time by the stop trigger sensor 56 is set, as in the first sheet, in such a manner as to unmask at the point of 10 mm in the sheet length L (STEP 2-1).

Next, when the start trigger sensor detection signal is asserted, the control unit 106 latches the signal of the start trigger sensor (processed) to mask detection by the start trigger sensor 55 until the predetermined set time set depending on the print speed and the sheet length L. When the signal of the start trigger sensor (processed) is asserted and lathed, the control unit 106 operates the encoder pulse counter, and the pulse counting unit 102 starts counting pulse signals. In the embodiment, the mask time of detection by the start trigger sensor 55 is set, as in the first sheet, in such a manner as to unmask at the point of 10 mm in the sheet length L (STEP 2-2).

Next, after a lapse of the set mask time of the stop trigger sensor 56, the control unit 106 asserts the stop trigger sensor edge detection valid signal, and enters the negate detection valid state in which the stop trigger sensor 56 can detect the passage of an edge of the sheet P (STEP 2-3).

When a negate at an edge of the sheet P is detected by the stop trigger sensor detection signal, the control unit 106 stores a count value of the encoder pulse counter in the encoder pulse count register (the count value at this point in time is n21). In order to judge whether the negate is the trailing edge of the sheet P or an edge of the punched hole, the control unit 106 sets, as the monitoring time, the time required by the diameter of the punched hole to pass the stop trigger sensor 56, and asserts and monitors the stop trigger sensor punched hole monitoring signal (STEP 2-4).

When the stop trigger sensor detection signal is asserted while the stop trigger sensor punched hole monitoring signal is being asserted, the control unit 106 judges that the negate detected in STEP 2-4 is not caused by the trailing edge of the sheet P but an edge of the punched hole. The control unit 106 then negates the stop trigger sensor punched hole monitoring signal, and stands by until the next negate of the stop trigger sensor detection signal is detected (STEP 2-5).

When the second negate is detected by the stop trigger sensor detection signal, the control unit 106 writes a count value of the encoder pulse counter over the value stored by the first negate edge in the encoder pulse count register, and stores the count value (the count value at this point in time is n22). In order to judge again whether the negate is the trailing edge of the sheet P or an edge of the punched hole, the control unit 106 sets, as the monitoring time, the time required by the diameter of the punched hole to pass the stop trigger sensor 56, and asserts and monitors the stop trigger sensor punched hole monitoring signal (STEP 2-6).

Next, after a lapse of the set mask time of the start trigger sensor 55, the control unit 106 asserts the start trigger sensor edge detection valid signal, and enters the negate detection valid state in which the start trigger sensor 55 can detect the passage of an edge of the sheet P (STEP 2-7).

When the negate at an edge of the sheet P is detected by the start trigger sensor detection signal, in order to judge whether the negate is the trailing edge of the sheet P or an edge of the punched hole, the control unit 106 sets, as the monitoring time, the time required by the diameter of the punched hole to pass the start trigger sensor 55, and asserts and monitors the start trigger sensor punched hole monitoring signal (STEP 2-8).

When the start trigger sensor detection signal is asserted while the start trigger sensor punched hole monitoring signal is being asserted, the control unit 106 judges that the negate detected in STEP 2-8 is not caused by the trailing edge of the sheet P but an edge of the punched hole. The control unit 106 then negates the start trigger sensor punched hole monitoring signal, and stands by until the next negate of the start trigger sensor detection signal is detected (STEP 2-9).

When the second negate is detected by the start trigger sensor detection signal, in order to judge again whether the negate is the trailing edge of the sheet P or an edge of the punched hole, the control unit 106 sets, as the monitoring time, the time required by the diameter of the punched hole to pass the start trigger sensor 55, and asserts and monitors the start trigger sensor punched hole monitoring signal (STEP 2-10).

If an edge of the sheet P is not detected within the monitoring time set again, the control unit 106 negates the stop trigger sensor punched hole monitoring signal, and judges that the edge of the sheet P detected in STEP 2-6 is the trailing edge of the sheet P. The control unit 106 then negates the signal of the stop trigger sensor (processed), and negates the stop trigger sensor edge detection valid signal. When the signal of the stop trigger sensor (processed) is negated, the encoder pulse count valid signal is negated to stop the encoder pulse counting (STEP 2-11).

After a lapse of the monitoring time set again, the control unit 106 negates the start trigger sensor punched hole monitoring signal, negates the signal of the start trigger sensor (processed), and negates the start trigger sensor edge detection valid signal. When the signal of the start trigger sensor (processed) is negated, the encoder pulse counter is cleared to the initial state (the initial value of the embodiment is zero) (STEP 2-12).

In this manner, in the sheet length measuring apparatus 100 according to the embodiment, an edge of the sheet P detected first is the leading edge of the sheet P. Accordingly, the sensors (the stop trigger sensor 56 and the start trigger sensor 55) are masked from that point. The sheet length L of the sheet P to be conveyed is known, and thus, the mask is removed from the sensors before the trailing edge of the sheet P reaches the detection position. When the passage of an edge of the sheet P is detected after unmasking, the edge may be a punched hole near the trailing edge of the sheet P. Accordingly, monitoring is performed for the time required by the punched hole to pass the sensors. If the passage of an edge of the sheet P is detected during the monitoring time, the detected edge of the sheet P is judged to be an edge of the punched hole, and it is a standby time until the passage of an edge of the sheet P is detected next. In contrast, if the passage of an edge of the sheet P is not detected during the monitoring time, the detected edge of the sheet P can be judged to be the trailing edge of the sheet P. Hence, even for special paper having a hole, such as punched paper, the leading and trailing edges of the sheet P can be detected without detecting an edge of the punched hole wrongly. Therefore, it is possible to enable even the measurement of a sheet length of special paper having a hole such as a punched hole, and to make the image scaling correction.

The image forming apparatus according to the embodiment can be applied any of a multifunction peripheral, a copier, a printer, a facsimile machine, and the like, which has at least two of a copy function, a printer function, a scanner function, and a facsimile function, as long as it is an image forming apparatus having a function of conveying a sheet-shaped recording medium and printing an image.

The present invention takes effect that even the sheet length of a special sheet medium having a hole such as a punched hole can be measured, and the image scaling correction can be made.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A sheet length measuring apparatus comprising:
a conveyance unit configured to convey a sheet medium;
a first detection unit that is provided downstream of the conveyance unit in a conveyance direction of the sheet medium to detect passage of an edge of the sheet medium;
a second detection unit that is provided upstream of the conveyance unit in the conveyance direction to detect passage of an edge of the sheet medium;
a conveyance amount measuring unit configured to measure a conveyance amount of the sheet medium conveyed by the conveyance unit;
a calculation unit configured to calculate a length of the sheet medium in the conveyance direction from detection results of the first and second detection units and the conveyance amount of the sheet medium;
a first control unit configured to, until a predetermined set time passes since the detection of the passage of a leading edge in the conveyance direction among edges of the sheet medium, regard detection of the passage of an edge of the sheet medium by each of the first and second detection units as not detected; and
a second control unit configured to, when passage of an edge of the sheet medium is not detected again before a lapse of a predetermined monitoring time since the second detection unit detects passage of an edge of the sheet medium being passing a detection position of the second detection unit, judge that the edge detected by the second detection unit is a trailing edge in the conveyance direction among the edges of the sheet medium.

2. The Sheet length measuring apparatus according to claim 1, wherein when passage of an edge of the sheet medium is detected at least one time or more before a lapse of the predetermined monitoring time since the second detection unit detects passage of an edge of the sheet medium being passing the detection position of the second detection unit, the second control unit judges that the edge lastly detected by the second detection unit is the trailing edge of the sheet medium.

3. The sheet length measuring apparatus according to claim 1, wherein the first control unit is configured to set the predetermined set time to an arbitrary time.

4. The sheet length measuring apparatus according to claim 3, wherein the first control unit is configured to set the predetermined set time to a time from after the detection of the passage of the leading edge of the sheet medium to before the passage of the trailing edge of the sheet medium.

5. The sheet length measuring apparatus according to claim 1, wherein the second control unit is configured to set the predetermined monitoring time to an arbitrary time.

6. The sheet length measuring apparatus according to claim 5, wherein the second control unit is configured to set the predetermined monitoring time to a time from after the detection of the passage of the edge of the sheet medium being passing the detection position of the second detection unit to the passage of a diameter of a hole portion provided in the sheet medium.

7. The sheet length measuring apparatus according to claim 1, wherein the first detection unit and the second detection unit are transmissive or reflective optical sensors.

8. An image forming apparatus comprising the sheet length measuring apparatus according to claim 1.
